(19) United States
(12) Patent Application Publication
(10) Pub. No.: US 2026/0000906 A1
(43) Pub. Date: Jan. 1, 2026

(54) ACTIVATION STRUCTURES IN PHOTOTHERAPEUTIC ILLUMINATION DEVICES AND RELATED METHODS

(71) Applicant: KNOW Bio, LLC, Morrisville, NC (US)

(72) Inventors: Thomas Matthew Womble, Wake Forest, NC (US); James Michael Lay, Apex, NC (US); P. Joseph DeSena, JR., Raleigh, NC (US); Riley Hook, Durham, NC (US); Sameer Tendulkar, Raleigh, NC (US)

(21) Appl. No.: 18/760,502

(22) Filed: Jul. 1, 2024

(51) Int. Cl. A61N 5/06

(52) U.S. Cl. CPC A61N 5/06; A61N 2005/0626; A61N 2005/0647; A61N 2005/0652; A61N 2005/0663

(57) ABSTRACT

Illumination devices for directing light on tissue to induce one or more biological effects, and more particularly activation structures in phototherapeutic illumination devices. Activation structures are integrated within phototherapeutic illumination devices and are configured to avoid electrical activation during shipping and/or between uses. Exemplary activation structures include various integrated sensors, such as accelerometers and proximity sensors. Signals from accelerometers may be used to trigger illumination devices to respond to subsequent signals from proximity sensors for beginning treatment. Accelerometers may be inactive or ignored during shipping until initial device charging.

### FIELD OF THE DISCLOSURE

The present disclosure relates generally to illumination devices for directing light on tissue to induce one or more biological effects and more particularly to activation structures in phototherapeutic illumination devices.

### BACKGROUND

Phototherapy, or light therapy, involves exposure of the body to light to induce biological effects and promote various health-related medical benefits. Advancements in therapeutic light treatments have demonstrated beneficial results for inactivating and/or reducing viral loads of infectious diseases. Phototherapeutic light treatments have also demonstrated other health-related benefits, including the promotion of hair growth, treatment of skin or tissue inflammation such as acne, promoting tissue or skin healing or rejuvenation, enhancing wound healing, pain management, reduction of wrinkles, scars, stretch marks, varicose veins, and spider veins, treating cardiovascular disease, treating erectile dysfunction, treating microbial infections, treating hyperbilirubinemia, and treating various oncological and non-oncological diseases and disorders including diseases induced by human papillomavirus (HPV).

Various mechanisms by which phototherapy has been suggested to provide therapeutic benefits include inactivating and inhibiting growth of microorganisms and pathogens, increasing circulation (e.g., by increasing the formation of new capillaries), stimulating the production of collagen, stimulating the release of adenosine triphosphate (ATP), enhancing porphyrin production, reducing excitability of nervous system tissues, modulating fibroblast activity, increasing phagocytosis, inducing thermal effects, stimulating tissue granulation and connective tissue phagocytosis, reducing inflammation, and stimulating acetylcholine release. Phototherapy has also been suggested to stimulate cells to produce nitric oxide, which may act as a signaling messenger, cytotoxin, antiapoptotic agent, antioxidant, and regulator of microcirculation. Nitric oxide is recognized to relax vasculature smooth muscles, dilate blood vessels, inhibit aggregation of platelets, and modulate T-cell mediated immune response. Generally, phototherapy shows promise for improving health and/or treating myriad medical conditions.

The art continues to seek improved phototherapeutic light treatments providing desirable health-related benefits while being capable of overcoming challenges associated with conventional phototherapeutic light treatments.

### SUMMARY

The present disclosure relates generally to illumination devices for directing light on tissue to induce one or more biological effects and more particularly to activation structures in phototherapeutic illumination devices. Activation structures are integrated within phototherapeutic illumination devices and are configured to avoid electrical activation during shipping and/or between uses. Exemplary activation structures include various integrated sensors, such as accelerometers and proximity sensors. Signals from accelerometers may be used to trigger illumination devices to respond to subsequent signals from proximity sensors for beginning treatment. Accelerometers may be inactive or ignored during shipping until initial device charging.

In one aspect, a phototherapy device comprises: an array of light-emitting devices; a control printed circuit board configured to control operation of the array of light-emitting devices; a first sensor; and a second sensor, the first sensor configured to provide a first signal that directs the control printed circuit board to respond to a second signal from the second sensor. In certain embodiments, the control printed circuit board is configured to electrically activate the array of light-emitting devices based on the second signal. In certain embodiments, the first sensor comprises an accelerometer, and the second sensor comprises a proximity sensor. In certain embodiments, the accelerometer is positioned on the control printed circuit board and the proximity sensor is positioned away from the control printed circuit board. The phototherapy device may further comprise a flexible substrate, wherein the control printed circuit board and the proximity sensor are on different portions of the flexible substate. In certain embodiments: the flexible substrate comprises a proximal surface and a distal surface that is opposite the proximal surface, the flexible substate being configured for positioning along a scalp of a user such that the proximal surface is closer to the scalp than the distal surface; the array of light-emitting devices is on the proximal surface; the control printed circuit board is on the distal surface; and the proximity sensor is on the proximal surface. The phototherapy device may further comprise a light-transmissive layer on the proximal surface of the flexible substrate, the light-transmissive layer configured to transmit at least some light emissions generated by the array of light emitting devices. The phototherapy device may further comprise an electronic connection port configured to receive an external power source, wherein the control printed circuit board is configured to ignore the first sensor until after the external power source is connected to the electronic connection port. In certain embodiments, the array of light-emitting devices is configured to generate light having a first peak wavelength in a range from 600 nanometers (nm) to 700 nm. In certain embodiments, the array of light-emitting devices is configured to generate light having a first peak wavelength in a range from 615 nm to 635 nm and a second peak wavelength in a range from 650 nm to 670 nm.

In another aspect, a method comprises: detecting a first signal from a first sensor within a phototherapy device; directing a control printed circuit board within the phototherapy device to respond to a second signal from a second sensor within the phototherapy device after receiving the first signal; and electrically activating an array of light-emitting devices after receiving the second signal. The method may further comprise connecting an external power source to charge the phototherapy device, wherein the phototherapy device is in a shipping mode before connecting the external power source, and the phototherapy device is configured to ignore the first sensor and the second sensor during the shipping mode. In certain embodiments, after connecting the external power source, the phototherapy device exits the shipping mode and begins a low power mode that will respond to the first sensor. In certain embodiments, after receiving the first signal during the low power mode, the phototherapy device exits the low power mode and begins monitoring for the second signal for a time period. The method may further comprise returning to the low power mode if the phototherapy device does not receive the second signal during the time period. In certain embodiments, the phototherapy device determines if a previous treatment was ended prematurely as a partial treatment, and resumes the partial treatment after receiving the second signal during the time period. In certain embodiments, the first sensor comprises an accelerometer, and the second sensor comprises a proximity sensor. The method may further comprise turning off the array of light-emitting devices in response to an over-temperature condition or an error condition. In certain embodiments, the array of light-emitting devices is configured to generate light having a first peak wavelength in a range from 600 nanometers (nm) to 700 nm. In certain embodiments, the array of light-emitting devices is configured to generate light having a first peak wavelength in a range from 615 nm to 635 nm and a second peak wavelength in a range from 650 nm to 670 nm.

In another aspect, any of the foregoing aspects individually or together, and/or various separate aspects and features as described herein, may be combined for additional advantage. Any of the various features and elements as disclosed herein may be combined with one or more other disclosed features and elements unless indicated to the contrary herein.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1A is an exploded perspective view of a phototherapy device embodying a wearable cap for delivering light to the scalp of a patient according to principles of the present disclosure.

FIG. 1B is a top perspective view of the phototherapy device of FIG. 1A.

FIG. 1C is a bottom perspective view of the phototherapy device of FIG. 1A.

FIG. 2A is an exploded perspective view of a flexible substrate and electronics assembly of the phototherapy device of FIG. 1A.

FIG. 2B is a perspective view of the assembled flexible substrate of FIG. 2A.

FIG. 2C is a bottom view of the assembled flexible substrate of FIG. 2A from the perspective of an opposing surface from the view of FIG. 2B.

FIG. 3 is a top view of a control printed circuit board from FIG. 2A.

FIG. 4 is a schematic diagram of an initial startup process flow for device startup of the phototherapy device of FIGS. 1A to 1C.

FIG. 5 is a schematic diagram of a process flow for assessing readiness of the phototherapy device of FIGS. 1A to 1C after exiting shipping mode.

FIG. 6 is a schematic diagram of a process flow during charging of the phototherapy device of FIGS. 1A to 1C.

FIG. 7 is a schematic diagram of a process flow for resuming a partial treatment for the phototherapy device of FIGS. 1A to 1C.

FIG. 8 is a schematic diagram of a process flow for resuming a full treatment for the phototherapy device of FIGS. 1A to 1C.

FIG. 9 is a schematic diagram of a process flow for treatment logic for the phototherapy device of FIGS. 1A to 1C.

FIG. 10 is a schematic diagram of a process flow for partial treatment logic for the phototherapy device of FIGS. 1A to 1C.

FIG. 11 is a schematic diagram of a process flow for over-temperature and/or error logic for the phototherapy device of FIGS. 1A to 1C.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Embodiments are described herein with reference to schematic illustrations of embodiments of the disclosure. As such, the actual dimensions of the layers and elements can be different, and variations from the shapes of the illustrations as a result, for example, of manufacturing techniques and/or tolerances, are expected. For example, a region illustrated or described as square or rectangular can have rounded or curved features, and regions shown as straight lines may have some irregularity. Thus, the regions illustrated in the figures are schematic and their shapes are not intended to illustrate the precise shape of a region of a device and are not intended to limit the scope of the disclosure. Additionally, sizes of structures or regions may be exaggerated relative to other structures or regions for illustrative purposes and, thus, are provided to illustrate the general structures of the present subject matter and may or may not be drawn to scale. Common elements between figures may be shown herein with common element numbers and may not be subsequently re-described.

The present disclosure relates generally to illumination devices for directing light on tissue to induce one or more biological effects, and more particularly to activation structures in phototherapeutic illumination devices. Activation structures are integrated within phototherapeutic illumination devices and are configured to avoid electrical activation during shipping and/or between uses. Exemplary activation structures include various integrated sensors, such as accelerometers and proximity sensors. Signals from accelerometers may be used to trigger or otherwise direct illumination devices to respond to subsequent signals from proximity sensors for beginning treatment. Accelerometers may be inactive or ignored during shipping until initial device charging.

Light, or phototherapeutic light, may be administered at one or more wavelengths with one or more corresponding doses to induce one or more biological effects for recipient tissue. Biological effects may include at least one of inactivating and inhibiting growth of one or more combinations of microorganisms and pathogens, including but not limited to viruses, bacteria, fungi, and other microbes, among others. Biological effects may also include one or more of upregulating and/or downregulating a local immune response, stimulating enzymatic generation of nitric oxide to increase endogenous stores of nitric oxide, releasing nitric oxide from endogenous stores of nitric oxide, inducing an anti-inflammatory effect, promoting increased blood flow in the brain for the treatment of dementia, promotion of hair growth, and/or modulation of foreign body responses (FBR) in tissues. In certain aspects, light may be referred to as nitric oxide modulating light to increase concentrations of unbound nitric oxide within living tissue. Light may also be administered at one or more wavelengths as a pre-exposure prophylaxis or a post-exposure prophylaxis to eliminate pathogens in or on tissue of the upper respiratory tract and/or amplify host defense systems. Embodiments of the present disclosure may be used to prevent and/or treat respiratory infections and other infectious diseases.

Wavelengths of light may be selected based on at least one intended biological effect for one or more of the targeted tissues and the targeted microorganisms and/or pathogens. In certain aspects, wavelengths of light may include visible light in any number of wavelength ranges based on the intended biological effect. Devices and related methods for light treatments include those that provide light doses for inducing biological effects on various targeted pathogens and/or targeted tissues with increased efficacy and reduced cytotoxicity. Light doses may include various combinations of irradiances, wavelengths, and exposure times, and such light doses may be administered continuously or discontinuously with a number of pulsed exposures.

Various wavelengths of visible light may be irradiated on human tissue with little or no impact on tissue viability. In certain embodiments, various wavelengths of visible light may elicit antimicrobial and/or anti-pathogenic behavior in corresponding tissues, including any of the aforementioned biological effects. For example, light with a peak wavelength in a range from 400 nanometers (nm) to 450 nm may inactivate microorganisms that are in a cell-free environment and/or inhibit replication of microorganisms that are in a cell-associated environment and/or stimulate enzymatic generation of nitric oxide, while also upregulating a local immune response in target tissue. In this regard, light with a peak wavelength in a range from 400 nm to 450 nm may be well suited for fighting invading viral and/or bacterial pathogens and corresponding diseases that may originate in the respiratory tract, including Orthomyxoviridae (e.g., influenza), common colds, coronavirida (e.g., coronavirus), picornavirus infections, tuberculosis, pneumonia, bronchitis, and sinusitis. In certain embodiments, red or near-infrared (NIR) light (e.g., peak wavelength range from 630 nm to 1,000 nm) may be useful to provide anti-inflammatory effects and/or to promote vasodilation. Anti-inflammatory effects may be useful in treating disorders, particularly microbial disorders that result in inflammation along the respiratory tract. In this regard, red light may be used as part of treatment protocols that reduce any tissue inflammation that may result from exposure to blue light, which may positively impact cell viability, thereby lowering cytotoxicity even further. A decrease in inflammation can be beneficial when treating viral infections, particularly when a virus can elicit a cytokine storm and/or inflammation can result in secondary bacterial infections. Accordingly, the combination of blue light, such as light at around 425 nm, and red light at one or more anti-inflammatory wavelengths, can provide a desirable combination of biological effects.

Aspects of the present disclosure are applicable to activation structures and related methods for a variety of phototherapeutic devices for a variety of target tissues. For exemplary purposes, wearable devices for delivering light energy to a scalp of a patient are described in greater detail below. It is understood that many of the principles disclosed for activation structures are applicable for other types of phototherapeutic illumination devices.

Before delving into specific details of various aspects of the present disclosure, an overview of various elements that may be included in exemplary phototherapeutic illumination devices for the scalp is provided for context. In certain aspects, illumination devices embody a wearable cap for positioning on a head of a patient during treatment. The illumination device includes one or more light-emitting elements for delivering light emissions to the scalp and one or more cameras for capturing images of the scalp.

Various types of light-emitting devices may be used for delivering light energy to the scalp of a patient. In certain embodiments, emissions may consist of non-coherent light, such as emissions generated by one or more light-emitting diodes (LEDs). According to principles of the present disclosure, arrangements of LEDs may provide improved scalp coverage with deeper penetration than coherent light sources. LEDs are arranged to emit overlapping cones of light that evenly cover the scalp for increased coverage of hair follicles. Coherent light sources, such as lasers, emit tightly focused beams that only cover discrete spots on the scalp. In certain embodiments, emissions of an illumination device may include combinations of coherent light and non-coherent light. Additionally, various aspects of the present disclosure, including the imaging capabilities described herein, are equally applicable to illumination devices with only coherent light sources.

In certain embodiments, multiple light-emitting devices of different peak wavelengths (e.g., having peak wavelengths differing by at least about 10 nanometers (nm), at least about 20 nm, at least about 30 nm, at least about 50 nm, at least about 75 nm, at least about 100 nm, or another threshold specified herein) may be provided. In certain embodiments, light of different peak wavelengths may be generated by different light-emitting devices contained in a single emitter package, wherein close spacing between adjacent emitters, such as LED chips, may provide integral color mixing. In certain embodiments, one or more arrays of light-emitting devices may be provided. For example, a first array of light-emitting devices may be configured to provide light of a first peak wavelength, and a second array of light-emitting devices may be configured to provide light of a second peak wavelength. In certain embodiments, an array of multi-emitter packages may be provided, wherein emitters within a single package may provide the same or different peak wavelengths. In certain embodiments, an array of solid state emitter packages may embody packages further including second, third, fourth, and/or fifth solid state emitters, such that a single array of solid state emitter packages may embody two, three, four, or five arrays of solid state emitters, wherein each array is arranged to generate emissions with a different peak wavelength.

In certain embodiments, an illumination device for delivering light energy to a scalp of a patient may include one or more light-emitting devices devoid of a wavelength conversion material. In other embodiments, one or more light-emitting devices may be arranged to stimulate a wavelength conversion material, such as a phosphor material, a fluorescent dye material, a quantum dot material, and a fluorophore material.

In certain embodiments, one or more light-emitting devices may be arranged to provide substantially monochromatic light. In certain embodiments, one or more light-emitting devices may include a spectral output having a full width at half maximum value of less than 25 nm (or less than 20 nm, or less than 15 nm, or in a range of from 5 nm to 25 nm, or in a range of from 10 nm to 25 nm, or in a range of from 15 nm to 25 nm). In certain embodiments, one or more light-emitting devices may be arranged to provide emissions having a peak wavelength in a range of from 400 nm to 900 nm, or in a range of from 500 nm to 900 nm, or in a range of from 500 nm to 800 nm, or in a range of from 600 nm to 700 nm, or in a range of from 620 nm to 670 nm.

In certain embodiments, at least one light-emitting device may be arranged to provide emissions having a peak wavelength in a range of from 615 nm to 645 nm (or from 615 nm to 635 nm), and at least one light-emitting device may be arranged to provide emissions having a peak wavelength in a range of from 645 nm to 670 nm (or from 650 nm to 670 nm). In certain embodiments, at least one first light-emitting device may be arranged to provide emissions having a peak wavelength of about 620 nm, and at least one second light-emitting device may be arranged to provide emissions having a peak wavelength of about 660 nm. Such combination of wavelengths and wavelength ranges may be useful to provide anti-inflammatory effects, to promote vasodilation, and/or to reduce or block dihydrotestosterone (DHT). Anti-inflammatory effects may be useful to promote wound healing, to reduce acne blemishes, to promote facial aesthetics, and/or to treat atopic dermatitis and other topical dermatological disorders. Vasodilation may also be beneficial to treat androgenic alopecia or other topical dermatological disorders.

While certain aspects of the present disclosure include light-emitting devices with wavelengths for treating alopecia, various aspects are also applicable to illumination devices for treating other conditions alone or in combination with alopecia. For example, illumination devices as described herein may include at least one light-emitting device configured to produce light in a wavelength range and flux that improves wound healing, reduces acne blemishes, and/or alters the presence, concentration, or growth of pathogens, bacteria, and/or other microbes in or on living mammalian tissue receiving the light. In certain embodiments, exemplary peak wavelength ranges include one or more combinations of peak wavelengths in a range from 400 nm to 1000 nm, or 400 nm to 450 nm, or 410 nm to 430 nm, or 600 nm to 1000 nm, or 615 nm to 645 nm, and/or 645 nm to 670 nm.

In certain embodiments, any suitable combination of peak wavelengths disclosed herein may be used for desired therapeutic effects (e.g., vasodilation, inflammation reduction, nitric oxide generation, nitric oxide release, and antimicrobial functions). In certain embodiments, a combination of wavelengths may be provided during the same time window, during overlapping but non-coincident time windows, or during non-overlapping time windows.

In certain embodiments, at least one first light emitter and at least one second light emitter (which may be embodied in a first array of light emitters and a second array of light emitters) may be arranged to provide different peak wavelengths selected from one of the following combinations (a) to (f): (a) the first peak wavelength is in a range of from 620 nm to 640 nm (or from 615 nm to 635 nm) and the second peak wavelength is in a range of from 650 nm to 670 nm; (b) the first peak wavelength is in a range of from 520 nm to 540 nm and the second peak wavelength is in a range of from 650 nm to 670 nm; (c) the first peak wavelength is in a range of from 400 nm to 420 nm (or from 410 nm to 430 nm) and the second peak wavelength is in a range of from 620 nm to 640 nm; (d) the first peak wavelength is in a range of from 400 nm to 420 nm (or from 410 nm to 430 nm) and the second peak wavelength is in a range of from 650 nm to 670 nm; (e) the first peak wavelength is in a range of from 400 nm to 420 nm (or from 410 nm to 430 nm) and the second peak wavelength is in a range of from 495 nm to 515 nm; and (f) the first peak wavelength is in a range of from 400 nm to 420 nm (or from 410 nm to 430 nm) and the second peak wavelength is in a range of from 520 nm to 540 nm.

In addition to various sources of light, the principles of the present disclosure are also applicable to one or more other types of directed energy sources. As used herein, a directed energy source may include any of the various light sources previously described, and/or an energy source capable of providing one or more of heat, infrared (IR) heating, resistance heating, radio waves, microwaves, soundwaves, ultrasound waves, electromagnetic interference, and electromagnetic radiation that may be directed to a target body tissue. Combinations of visual and non-visual electromagnetic radiation may include peak wavelengths in a range from 180 nm to 4,000 nm. Illumination devices as disclosed herein may include a light source and another directed energy source capable of providing directed energy beyond visible light. In other embodiments, the other directed energy source capable of providing directed energy beyond visible light may be provided separately from illumination devices of the present disclosure.

In certain embodiments, one or more light-emitting devices may provide a fluence of at least 1 joule per square centimeter (J/cm²), at least 3 (J/cm²), or at least 5 (J/cm²) when energized to emit light. In certain embodiments, one or more light-emitting devices may provide a radiant flux in a range of from 1 milliwatts per square centimeter (mW/cm²) to 60 mW/cm². In certain embodiments, one or more light-emitting devices may be arranged to provide substantially steady state light. In certain embodiments, one or more light-emitting devices may be arranged to provide multiple discrete pulses of light.

In certain embodiments, light-emitting devices may be arranged on one or more flexible substrates configured to conform to the shape of a wearable cap for positioning on the head of a patient. The flexible substrate may comprise a flexible printed circuit board (FPCB) supporting at least one light-emitting device. In certain embodiments, a FPCB may include a polyimide-containing layer and at least one layer of copper or another electrically conductive material. In certain embodiments, a light-transmissive layer (e.g., an encapsulant or lens) may be arranged to cover and/or arranged in contact with at least a portion of a FPCB and any light emitter(s) supported thereon. An exemplary material for the light-transmissive layer is silicone, which may be applied by any suitable means such as molding, dipping, spraying, dispensing, printing, or the like. In certain embodiments, substantially all surfaces (e.g., front and back surfaces) of a FPCB may be covered with encapsulant material. In certain embodiments, the total thickness of an encapsulated flexible LED including embedded light emitters may be in a range of 1 millimeter (mm) to 5 mm, or in a range of from 1 mm to 3 mm, not including standoffs. In certain embodiments, the FPCB comprises a flexible polymer film, polyester (PET), polyimide (PI), polyethylene naphthalate (PEN), polyetherimide (PEI), fluoropolymers (FEP), copolymers, etc.

In certain embodiments, at least one standoff is configured to be arranged between the FPCB and the scalp of the patient, with the at least one standoff including a standoff height that exceeds a height of emitters supported by the FPCB. Preferably, the at least one standoff comprises a light-transmissive material such as silicone, PET, polyethylene terephthalate glycol (PET-G), etc. Various steps of forming an encapsulated FPCB with standoffs may include defining electrical traces on the FPCB; mounting, forming or otherwise affixing one or more light-emitting elements on the FPCB; forming standoffs or standoff portions; and encapsulating various structures including the light-emitting elements, the FPCB, and optionally encapsulating standoffs or standoff portions. The order of the preceding steps may be altered, and in certain embodiments, portions or the entirety of at least some standoffs may be devoid of encapsulant.

In certain embodiments, standoffs or standoff portions may be molded, placed, formed, printed, adhered, or otherwise applied to a face of a FPCB prior to encapsulation, and the standoffs or standoff portions may thereafter be partially or fully encapsulated together with one or more light-emitting elements and one or more portions of the FPCB. In other embodiments, standoffs or standoff portions may be placed, formed, printed, adhered, or otherwise applied to a face of a FPCB after the FPCB and light-emitting elements have been encapsulated. In various embodiments, standoffs or standoff portions may be formed concurrently with an encapsulation process for the light transmissive layer, such as by molding, printing, spraying, or other deposition methods.

Standoffs or standoff portions may be formed by cross-linkable materials selectively applied or formed along regions of a FPCB, and such materials may be activated by appropriate means (e.g., heat, photonic energy, chemical activation, or the like) before, during, or after an encapsulation step.

In certain embodiments, standoff height, standoff shape, light-emitting element spacing, and light element optical distribution may be selected to permit adjacent light-emitting elements to provide an overlapping beam pattern on a scalp of a patient. In certain embodiments, an array of multiple standoffs may be formed on, in, or over an encapsulant material. In certain embodiments, each standoff within an array has substantially the same size, shape, and/or durometer. In other embodiments, different standoffs within an array may include different sizes, shapes, and/or durometers. In certain embodiments, one or more standoffs may include suitable shapes and/or materials to provide light-focusing utility, light-diffusing utility, and/or light-scattering utility. In certain embodiments, one or more standoffs may include one or more wavelength conversion materials (e.g., phosphors, quantum dots, fluorophores, or the like) and provide wavelength conversion utility. In certain embodiments, one or more standoffs may include suitable shapes and/or materials to provide light reflection utility. In certain embodiments, one or more standoffs may be placed apart from one or more light-emitting elements. In other embodiments, one or more standoffs may be intentionally placed on or over one or more light-emitting elements, with the standoff(s) serving to transmit, shape, and/or otherwise affect light received from one or more light-emitting elements.

Illumination devices for delivering light energy to a scalp of a patient may include a FPCB with multiple interconnected panels and a plurality of bending regions defined in and between the multiple panels to allow the FPCB to provide a concave shape to cover at least a portion of a cranial vertex of a patient. In certain embodiments, the FPCB is formed with a shape that includes various extensions and/or flaps that conform to a concave shape with sharp bending regions. In certain embodiments, openings are provided between portions of adjacent panels to permit transport of heat and fluids (e.g., perspiration). In certain embodiments, a fabric covering may be arranged to cover the FPCB, with the fabric covering preferably being breathable to permit transport of heat and fluid transport (e.g., evaporation of sweat). In certain embodiments, the fabric covering may include an adjustable closure arranged to permit an opening circumference of the fabric covering to be adjusted. If the FPCB is contained within the fabric covering, then adjustment of the closure may selectively compress a portion of the FPCB and therefore also permit an opening circumference of the FPCB to be adjusted. In certain embodiments, the FPCB and the fabric covering are arranged to accommodate outward expansion and inward contraction to permit standoffs of the FPCB to contact the scalp of the patient.

In certain embodiments, the FPCB may form a plurality of curved panels projecting generally outwardly and downwardly from a central frame to substantially conform to portions of the cranium. Gaps may be provided between portions of curved panels to accommodate outward expansion and inward contraction, and to enable dissipation of heat generated by the at least one light-emitting device associated with the FPCB.

In certain embodiments, a flexible shaping member having a generally concave interior may be arranged to receive a FPCB. The flexible shaping member may be provided between the FPCB and fabric covering to accommodate outward expansion and inward contraction to permit the plurality of standoffs to contact the scalp of the patient. In certain embodiments, a flexible shaping member may be fabricated from a suitable polymeric material.

In certain embodiments, an illumination device may include an electronics housing. The electronics housing may include driver circuitry (or at least a portion of driver circuitry) configured to energize at least one light-emitting device for impingement of light on the scalp of a patient. In certain embodiments, the electronics housing may include one or more of a user interface, sensory interface, charging interface, data interface, signal input, signal output, and/or display elements. In certain embodiments, an energy storage device (e.g., a battery) may be retained by a battery holder pivotally (or otherwise movably) coupled to the electronics housing. Such movable coupling may permit relative movement between the battery holder and electronics housing to permit the phototherapy device to accommodate a variety of patients having different head sizes and shapes.

In certain embodiments, operation of an illumination device as disclosed herein may be responsive to one or more signals generated by one or more sensors or other elements. Various types of sensors are contemplated, including temperature sensors, photosensors, image sensors, proximity sensors, pressure sensors, chemical sensors, biosensors, accelerometers, moisture sensors, oximeters, current sensors, voltage sensors, and the like. Other elements that may affect impingement of light and/or operation of a device as disclosed herein include a timer, a cycle counter, a manually operated control element, a wireless transmitter and/or receiver (as may be embodied in a transceiver), a laptop or tablet computer, a mobile phone, or another portable digital device. Wired and/or wireless communication between a device as disclosed herein and one or more signal generating or signal receiving elements may be provided.

In certain embodiments, an illumination device as disclosed herein may be configured to prevent unauthorized usage beyond an authorized number of treatment cycles. For example, a number of treatment cycles of the device may be incremented and stored in a counter or other memory element. When the number of treatment cycles reaches a predetermined limit, operation of the illumination device may be reversibly or irreversibly disabled. In certain embodiments, when the number of treatment cycles reaches a predetermined limit, a signal may be communicated to a user to notify the user that a predetermined limit of a number of treatment cycles has been reached, and a user may be prompted to either (i) purchase a new device or component thereof, or (ii) purchase the ability to continue using the device for a specified number of additional cycles or for a specified additional time period. In certain embodiments, one or more signals relating to cycle usage and/or enabling a user to purchase additional usage may be communicated via wired or wireless means. In certain embodiments, a user may download an application for use on a personal computer, a tablet computer, a mobile phone, or another portable digital device, and the application may provide cycle usage information and/or permit the user to purchase additional cycles or purchase additional usage time to continue using the device.

In certain embodiments, upon detection of a specified number of uses of the device, a light-emitting device may be configured to produce a disabling signal adapted to irreversibly disable the device to prevent further operation of the device. In certain embodiments, the disabling signal may include at least one of a voltage spike and a current spike arranged to damage at least one circuit element. In certain embodiments, a light-emitting device includes a power supply circuit arranged to provide at least one conditioned power signal for use by a microcontroller of the device and/or the at least one light-emitting device, and a disabling signal may be adapted to irreversibly disable at least one element of the power supply circuit. In certain embodiments, at least one fusible link may be arranged in electrical communication with the at least one light-emitting device, and a disabling signal may be adapted to open the at least one fusible link to prevent current from being supplied to the at least one light-emitting device. In certain embodiments, at least one fusible link may be arranged in electrical communication with at least one light emitter and/or a light emitter driver circuit.

In certain embodiments, impingement of light on living tissue and/or operation of an illumination device as disclosed herein may be responsive to one or more temperature signals. For example, a temperature condition may be sensed on or proximate to a FPCB; at least one signal indicative of the temperature condition may be generated; and operation of a device for delivering light energy to a scalp of a patient may be controlled responsive to the at least one signal. Such control may include initiation of operation, deviation (or alteration) of operation, or termination of operation of light-emitting elements. In certain embodiments, thermal foldback protection may be provided at a threshold temperature (e.g., >42° Celsius) to prevent a user from experiencing burns or discomfort. In certain embodiments, thermal foldback protection may trigger a light-emitting device to terminate operation, reduce current, or change an operating state in response to receipt of a signal indicating an excess temperature condition.

In certain embodiments, a proximity sensor may be arranged proximate to a portion of a FPCB to determine when a FPCB is proximate to a surface (e.g., scalp) to be illuminated and used for safety of a patient by reducing flux when not proximate to the surface.

In certain embodiments, a device for delivering light energy to a scalp of a patient may include a user-perceptible visible signaling element (e.g., one or more lights, a LED display, an alphanumeric display, mobile app, or the like) arranged to generate a visible signal and/or a user-perceptible audible signaling element (e.g., a speaker, a buzzer, an alarm generator, or the like) arranged to generate an audible signal. In certain embodiments, at least one of the visible signal and the audible signal is indicative of an operating status or a charging status of the device. In certain embodiments, at least one of the visible signal and the audible signal is indicative of a count of operating cycles of the device.

In certain embodiments, a device for delivering light energy to a scalp of a patient as disclosed herein may include a memory element to store information indicative of one or more sensor signals. Such information may be used for detecting device usage, assessing patient status, assessing patient improvement, and assessing function of the device. In certain embodiments, information indicative of one or more sensor signals and/or captured data and/or images may be transmitted via wired or wireless means (e.g., via Bluetooth, WiFi, Zigbee, or another suitable protocol) to a mobile phone, a computer, a data logging device, or another suitable device that may optionally be connected to a local network, a wide-area network, a telephonic network, or other communication network. In certain embodiments, a data port (e.g., USB-C, micro USB or other type) may be provided to permit extraction or interrogation of information contained in a memory.

According to principles of the present disclosure, activation structures that are integrated within phototherapeutic illumination devices are disclosed that avoid electrical activation during shipping and/or between uses. An exemplary activation structure may include a first sensor, such as an accelerometer, and a second sensor, such as a proximity sensor, integrated within an illumination device. In certain embodiments, the accelerometer is configured to be inactive or ignored by control circuitry until a user charges the illumination device. The illumination device may be provided with firmware configured to ignore the accelerometer until a charging cable is connected. In this manner, the illumination device may remain in an inactive state, or a low power mode, during shipping and storage. Once a user unpacks the illumination device and connects the charging cable, the device may leave low power mode and begin responding to detection by the accelerometer. For example, when the accelerometer detects motion after charging, the illumination device may then respond to signals from one or more proximity sensors. When a proximity sensor detects placement of the illumination device for use, such as on the user's scalp, phototherapeutic treatment may begin. In certain embodiments, if no signal is provided by the proximity sensor after a time period has elapsed from motion detection by the accelerometer, then the illumination device may return to low power mode. In this manner, illumination devices as described herein provide an element of sophistication for activation over simple on/off power buttons, while also avoiding the unnecessary complexity of applications run by mobile devices and/or computers.

The following embodiments are described in the context of first sensors comprising accelerometers and second sensors comprising proximity sensors in the context of phototherapy devices for delivering light emissions to a scalp of a patient. The principles disclosed are applicable to any phototherapeutic illumination device with first and second sensors, where control circuitry is configured to respond to signals from the second sensor after receiving a signal from the first sensor. As described above, various types of sensors for the first and second sensors are contemplated, including temperature sensors, photosensors, image sensors, proximity sensors, pressure sensors, chemical sensors, biosensors, accelerometers, moisture sensors, oximeters, current sensors, voltage sensors, and combinations thereof.

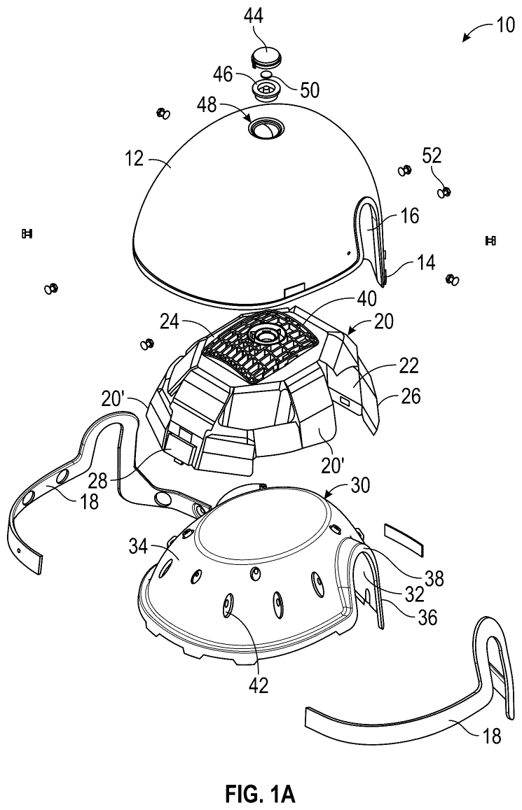

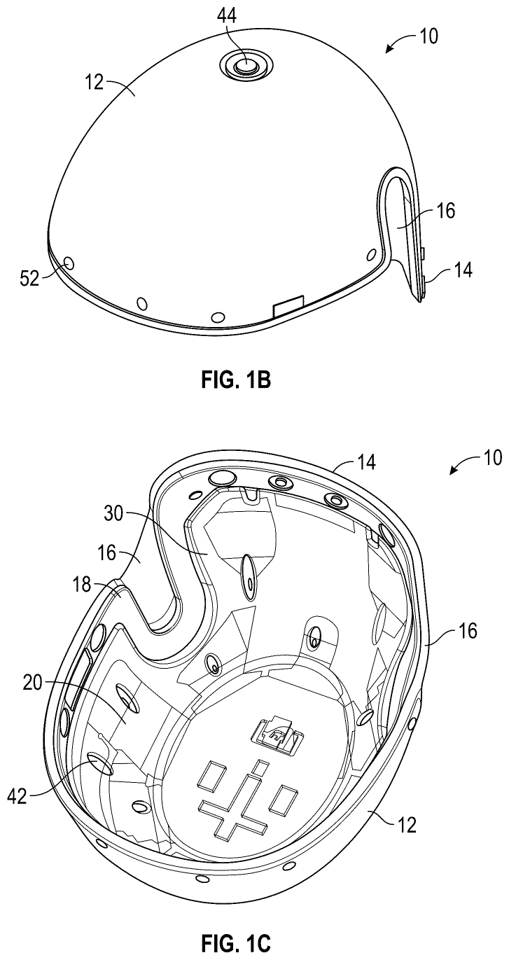

FIG. 1A is an exploded perspective view of a phototherapy device 10 embodying a wearable cap for delivering light to the scalp of a patient according to principles of the present disclosure. FIG. 1B is a top perspective view of the phototherapy device 10 of FIG. 1A. FIG. 1C is a bottom perspective view of the phototherapy device 10 of FIG. 1A.

The phototherapy device 10 includes a cap 12 that may be formed of one or more fabrics or materials, such as cotton, open or closed cell polyethylene foam, polyester, and rayon, among others. The phototherapy device 10 may be formed in a variety of different shapes and sizes depending on the head dimensions of the patient. In certain embodiments, the cap 12 is a flexible cap with a stretchable material for accommodating flexibility of the phototherapy device 10 for a variety of head sizes. The phototherapy device 10 is generally sized and shaped to receive an upper portion of a head of a patient. In certain embodiments, the phototherapy device 10 includes a posterior extension 14 configured to cover the lower rear portion of a patient's head (e.g., the nape, posterior hairline, occipital protuberance, and/or proximate thereto, etc.). Proximate to the posterior extension 14 and towards a center of the phototherapy device 10, one or more flex arcs 16 are positioned to allow the posterior extension 14 to more easily flex outward to accommodate varying head sizes. The flex arcs 16 may embody a stretchable material that resides within a cutout portion of the phototherapy device 10 to accommodate outward and/or inward flexibility. In certain embodiments, a padding 18, such as foam padding, may be provided within the cap 12 for comfort.

The phototherapy device 10 may include a flexible substrate 20, such as a FPCB. As best illustrated in the exploded view of FIG. 1A, the flexible substrate 20 may be formed with a shape that includes various interconnected flaps 20′ or panels that promote flexibility for conforming to the concavity of the phototherapy device 10 and for any inward or outward expansion based on a user's head size. The flexible substrate 20 includes a proximal surface 22 (which may variously be referred to as a bottom surface, lower surface, inner surface, inside surface, or surface proximate to the patient) and a distal surface 24 (which may variously be referred to as a top surface, upper surface, outer surface, outside surface, or surface further from the patient). As illustrated, the distal surface 24 faces the cap 12. The flexible substrate 20 may form a concavity generally sized and shaped to receive at least an upper portion of the head of a patient and may include a posterior extension 26 configured to cover the lower rear portion of a patient's head (e.g., the nape, posterior hairline, occipital protuberance, and/or proximate thereto, etc.). As will be described later in greater detail, an array of light-emitting devices, such as LEDs, are provided across the proximal surface 22 in positions to direct light toward a user. In certain embodiments, a support pad 28 and an underlying proximity sensor may be located on one or more portions of the flexible substrate 20. The support pad 28 is on the distal surface 24 to provide padding with the cap 12, and the corresponding proximity sensor may be located on the proximal surface 22 in a position that is registered with the support pad 28. By way of example, the support pad 28 and corresponding proximity sensor are located near a front of the phototherapy device 10 proximate a forehead position of the user when in use.

A light-transmissive layer 30, such as a flexible lens, includes a proximal surface 32 (e.g., inner surface, inside surface, surface proximate to the user) and a distal surface 34 (e.g., outer surface, outside surface, surface further from the user). The light-transmissive layer 30 forms a concavity generally sized and shaped to the head of a user, and may include a posterior extension 36 configured to cover the lower part of a back of a patient's head (e.g., the nape, posterior hairline, occipital protuberance, and/or proximate thereto, etc.). Proximate the posterior extension 36 (but towards the center of the light-transmissive layer 30), the light-transmissive layer 30 may include flex arcs 38 on opposing sides, which allow the posterior extension 36 to more easily flex outward to accommodate varying head sizes. The light-transmissive layer 30 may be molded and may have a thickness of approximately 0.02 inches (in). to 0.06 in. In certain embodiments, the light-transmissive layer 30 includes a plurality of lenses with a lens density in a range of from about 10 to about 80 lenses per inch (LPI), or from about 20 to about 60 LPI, or from about 30 to about 50 LPI, or about 40 LPI, although other dimensions may be used. In certain embodiments, the light-transmissive layer 30 may have a lenticular surface for directing light therethrough. Depending on the materials used, if the light-transmissive layer 30 has a thickness smaller than about 0.02 in., then undesirable wrinkling or crinkling may result, and if the light-transmissive layer 30 has a thickness greater than 0.06 in., it may be insufficiently flexible or stretchable to accommodate head sizes of different patients. As described herein, the light-transmissive layer 30 may embody an encapsulant or an encapsulant material as described above.

The flexible substrate 20 is positioned between the cap 12 and the light-transmissive layer 30. More specifically, the flexible substrate 20 is positioned within the concavity of the cap 12 such that the flexible substrate distal surface 24 is proximate the cap 12. The light-transmissive layer 30 is positioned within the concavity of the flexible substrate 20 such that the distal surface 34 of the light-transmissive layer 30 is positioned proximate the proximal surface 22 of the flexible substrate 20. In this manner, when the cap 12, the flexible substrate 20, and the light-transmissive layer 30 are assembled together, the concavities and peripheral edges thereof may be generally aligned with one another. In the same manner, the cap posterior extension 14, the flexible substrate posterior extension 26, and the light-transmissive layer posterior extension 36 are all generally aligned with one another.

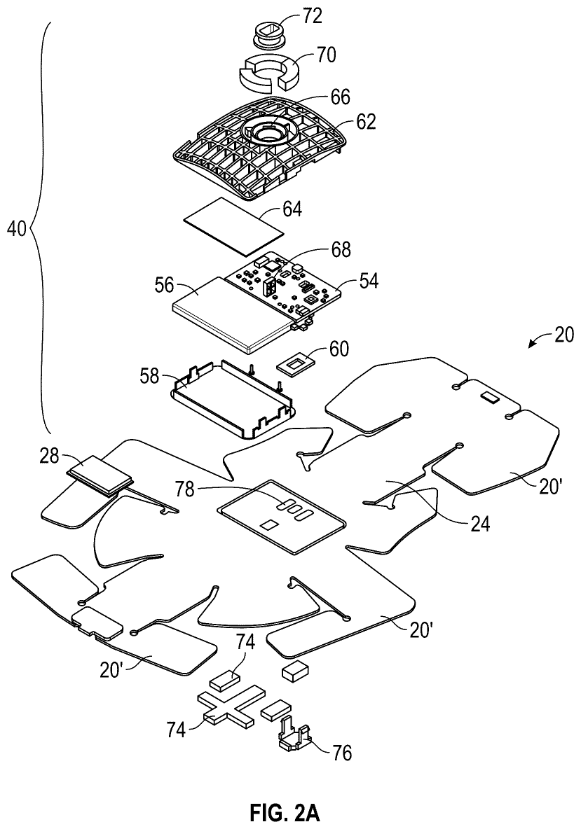

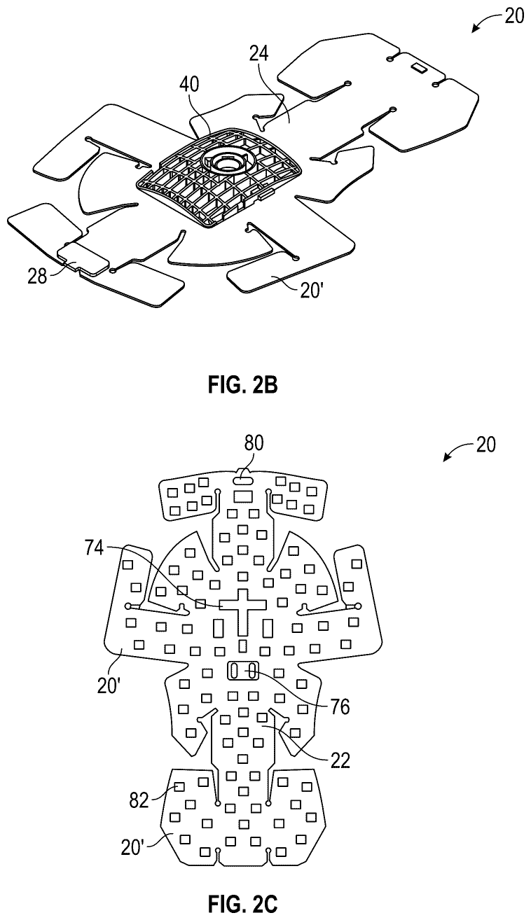

An electronics assembly 40 may be located on the distal surface 24 of the flexible substrate 20. As will be described later in association with FIG. 2, the electronics assembly 40 may be attached to the distal surface 24, and the electronics assembly 40 may include a control printed circuit board with an accelerometer and driver circuitry for controlling operation of the phototherapy device 10.

A plurality of standoffs 42 may include top standoffs positioned at or along the distal surface 34 of the light-transmissive layer 30. In certain embodiments, the standoffs 42 may be attached to (e.g., by adhesive) or may be integrally formed with the light-transmissive layer 30 (e.g., by molding the standoffs 42 concurrently with the light-transmissive layer 30). More specifically, for each standoff 42, a standoff proximal end contacts and extends from the light-transmissive layer distal surface 34, and a standoff distal end may contact the flexible substrate proximal surface 22. The height of the standoffs 42 may be greater than a height of the light-emitting devices to prevent the light-transmissive layer 30 from contacting the light-emitting devices. In this manner, the standoffs 42 maintain a minimum distance between the light-transmissive layer 30 and the flexible substrate 20.

The cap 12 may include a seal plug 44 removably attached to and covering an electronic connection port 46 at a top of the cap 12. The electronic connection port 46 is mechanically attached to an electronics receptacle 48 arranged at a top of the cap 12. The seal plug 44 covers and protects the electronic connection port 46 from damage when not in use. The electronics receptacle 48 may form an opening on an interior surface of the cap 12 facing the flexible substrate 20 for receiving the electronics assembly 40. For example, the electronics receptacle 48 may be mechanically attached and secured to the electronics subassembly to prevent relative movement therebetween. The electronic connection port 46 (with the electronics receptacle 48) may provide mechanical and/or electronic connectivity between the phototherapy device 10 and an electronic device (e.g., computer, smartphone, etc.) external to the phototherapy device 10. The electronic connection port 46 may also embody an electronic connector (e.g., power cord, USB cord, etc.) to receive electrical power and/or electronic data (e.g., operational parameters). In certain embodiments, wireless communication may be provided between the phototherapy device 10 and an external electronic device. In certain embodiments, a battery operatively coupled to the flexible substrate 20 may be inductively charged (e.g., wirelessly charged). An instruction manual label 50 may be positioned between the seal plug 44 and the electronic connection port 46. In certain embodiments, a number of mechanical connectors 52, such as rivets, may be employed to mechanically secure various elements together when assembled.

FIG. 2A is an exploded perspective view of the flexible substrate 20 and electronics assembly 40 of the phototherapy device 10 of FIG. 1A. FIG. 2B is a perspective view of the assembled flexible substrate 20 of FIG. 2A. The views of FIGS. 2A and 2B are from the perspective of the distal surface 24 of the flexible substrate 20. FIG. 2C is a bottom view of the assembled flexible substrate 20 of FIG. 2A from the perspective of the proximal surface 22.

As illustrated, a control printed circuit board 54 is positioned within the electronics assembly 40. A power source 56, such as a rechargeable battery, may be positioned adjacent the control printed circuit board 54. In certain embodiments, a power source cover 58 may be arranged to enclose at least a portion of the power source 56. When assembled on the distal surface 24, a foam connector pad 60 may provide support between the control printed circuit board 54 and the distal surface 24. The electronics assembly 40 may further include a support structure 62 that encloses portions of the power source 56 and the control printed circuit board 54. In certain embodiments, a double-sided tape 64 may be employed to adhere the power source 56 to the support structure 62. An opening 66 of the support structure 62 is arranged to receive a connector 68, such as a USB connector, of the control printed circuit board 54. A foam locking tab 70 and connector gasket 72 may be positioned at the opening 66. In this manner, external power for charging and other communication signals may be routed from the electronic connection port 46 of FIG. 1A to the control printed circuit board 54 via the connector gasket 72, opening 66, and connector 68. One or more standoff pads 74 may be positioned on the proximal surface 22 to provide spacing with the light-transmissive layer 30 of FIG. 1A. As best depicted in FIG. 2C, a proximity sensor 80 is positioned on the proximal surface 22 and registered with the support pad 28 of FIG. 2B. As will be later described in greater detail, the proximity sensor 80 is configured to relay a signal to the control printed circuit board 54 that the phototherapy device 10 is positioned on a user's scalp for treatment. An array of light-emitting devices 82, such as LEDs, are provided across the proximal surface 22 of the flexible substrate 20 in positions to provide broad coverage of light emissions.

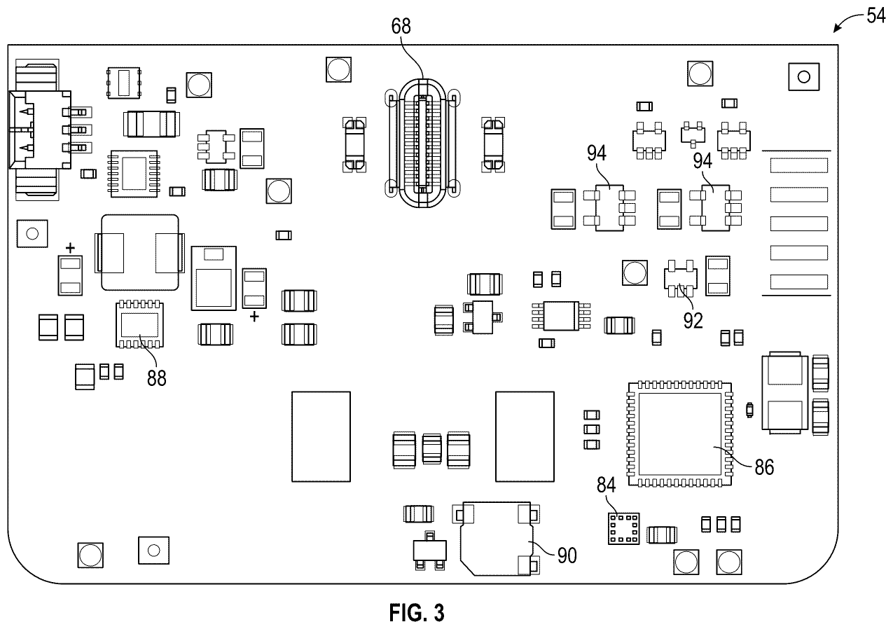

FIG. 3 is a top view of the control printed circuit board 54 of FIG. 2A. The control printed circuit board 54 may include an accelerometer 84 configured to detect movement. In other embodiments, the accelerometer 84 may be positioned away from the control printed circuit board 54. As will be described in greater detail below, the accelerometer 84 is configured to be activated after initial charging of the phototherapy device 10 of FIGS. 1A to 1C. Once active, the accelerometer 84 is configured to signal the occurrence of motion to the control printed circuit board 54. After receiving a motion signal from the accelerometer 84, the control printed circuit board 54 may be directed to now respond to any signals from the proximity sensor 80 of FIG. 2C. In certain embodiments, the control printed circuit board 54 may begin a time period of waiting for a signal from the proximity sensor 80 of FIG. 2C that the illumination device has been positioned for treatment. As further illustrated in FIG. 3, the control printed circuit board 54 may include a microcontroller 86 and a driver 88 for the light emitting devices 82 of FIG. 2C. Once the accelerometer 84 is active and a signal is received by the proximity sensor 80 of FIG. 2C, the microcontroller 86 and driver 88 may initiate treatment. In certain embodiments, the control printed circuit board 54 may further include a speaker 90, such as a loudspeaker, for providing audible signals for a user. The control printed circuit board 54 may also include a temperature sensor 92 for over-temperature prevention. For example, if the temperature sensor 92 detects a temperature that exceeds a threshold, treatment may be altered to avoid a potentially harmful over-temperature condition. For example, firmware of the microcontroller 86 and/or other elements of the control printed circuit board 54 may reduce output of the light-emitting devices 82 or even turn them off. In certain embodiments, additional hardware of the control printed circuit board 54, such as one or more temperature switches 94 may provide a redundant feature to the firmware. In such embodiments, the firmware may operate by reducing output in response to detection of high temperatures. In the event a certain temperature threshold is reached despite the actions of the firmware, the temperature switches 94 may then shut off operation. As indicated above, the firmware approach may be sufficient to avoid over-temperature conditions in various applications, wherein the temperature switches may be omitted.

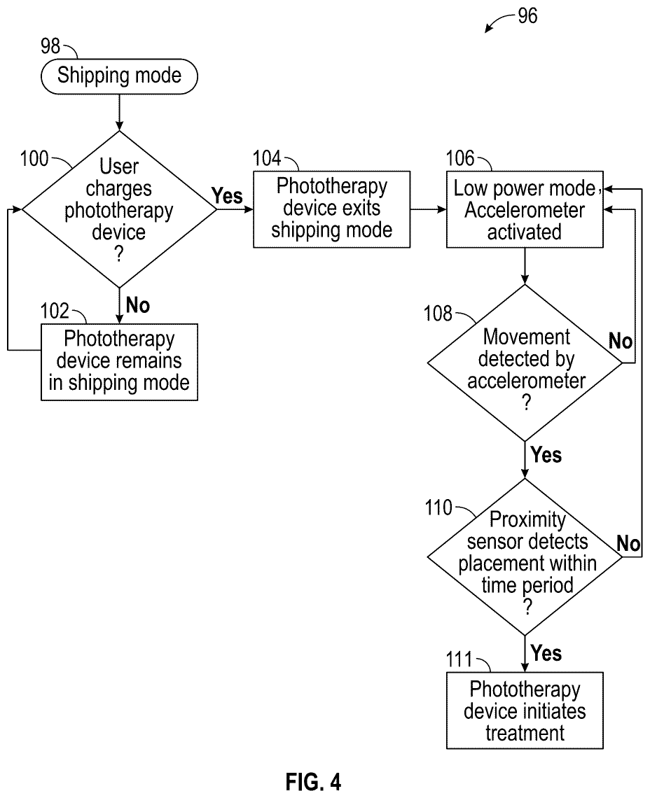

FIGS. 4 to 11 provide schematic diagrams of process flows for operation of the phototherapy device 10 of FIGS. 1A to 1C, including the flexible substrate 20 of FIGS. 2A to 2C, and the control printed circuit board 54 of FIG. 3. The process flows depicted in FIGS. 4 to 11 may represent steps that are controlled by firmware of the control printed circuit board 54, such as the microcontroller 86 and corresponding circuitry. In various embodiments, the individual steps represented in each of FIGS. 4 to 11 provide various options for partial or full process flows that may be implemented for the phototherapy device 10 of FIGS. 1A to 1C. In practice, exemplary phototherapy devices may implement any of the steps depicted in FIGS. 4 to 11 alone or in combination with other steps depicted in FIGS. 4 to 11.

FIG. 4 is a schematic diagram of an initial startup process flow 96 for device startup of the phototherapy device 10 of FIGS. 1A to 1C. As illustrated, after manufacturing and during shipping, the phototherapy device may be placed in an inactive shipping mode (step 98), where any signals from the accelerometer and the proximity sensor are ignored. Within the packaging of the phototherapy device, instructions are provided that direct the user to plug in the phototherapy device to an external power source for charging (step 100). Until plugged in for charging, the phototherapy device remains in shipping mode (step 102), and any movement detected by the accelerometer during shipping mode is ignored. After being plugged in for charging, the phototherapy device (e.g., the control printed circuit board) may exit shipping mode (step 104) and begin a low power mode (step 106). During low power mode, the accelerometer may be activated and the phototherapy device begins monitoring for a signal from the accelerometer. Once movement is detected by the accelerometer (step 108), the signal from the accelerometer is sent to the control printed circuit board for directing the control printed circuit board to respond to a signal from the proximity sensor. In this regard, the phototherapy device exits low power mode and begins monitoring for a signal from the proximity sensor. In certain embodiments, monitoring for a signal from the proximity sensor may take place for a certain time period, such as two minutes, or five minutes, or ten minutes (step 110). If a signal from the proximity sensor is detected during the time period, the phototherapy treatment may be initiated (step 111). If a signal from the proximity sensor is not detected during the time period, the phototherapy device may return to low power mode (step 106) and begin monitoring for a next signal from the accelerometer (step 108).

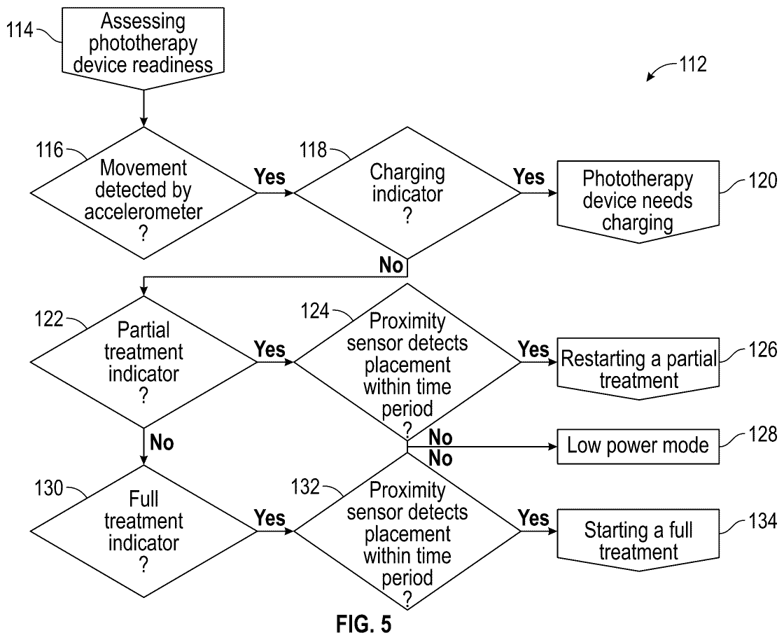

FIG. 5 is a schematic diagram of a process flow 112 for assessing readiness of the phototherapy device 10 of FIGS. 1A to 1C after exiting shipping mode. During an assessing phototherapy device readiness state (step 114), the phototherapy device may remain in a low power mode while waiting for a signal from the accelerometer. If movement is detected by the accelerometer (step 116), the logic progresses to determine if charging is required before treatment. If charging is required, a charging indicator (step 118), such as a pulsing indicator light of a first color, will prompt the user to plug the phototherapy device in for charging (step 120). If charging is not required, the phototherapy device will determine whether a previous treatment was ended prematurely as a partial treatment. If so, then a partial treatment indicator, such as a pulsing light of a second color that is different from the first color, may be displayed for a user (step 122). The phototherapy device may then begin monitoring for a signal from the proximity sensor that the phototherapy device is positioned in place for treatment (step 124). If a signal is received by the proximity sensor within a set time period as described above for FIG. 4, then the previous partial treatment may be restarted (step 126). If no signal is detected by the proximity sensor, the phototherapy device may return to low power mode (step 128) and wait for a next movement detected by the accelerometer. If no partial treatment is indicated, the device may then provide a full or standard treatment indicator, such as a solid light of the second color, to the user (step 130). The phototherapy device may then begin monitoring for a signal from the proximity sensor that the phototherapy device is positioned in place for treatment (step 132), and progress to initiating a full treatment (step 134) based on the signal from the proximity sensor. If no signal from the proximity sensor is detected during the set time period, the phototherapy device may return to low power mode (step 128) as described above.

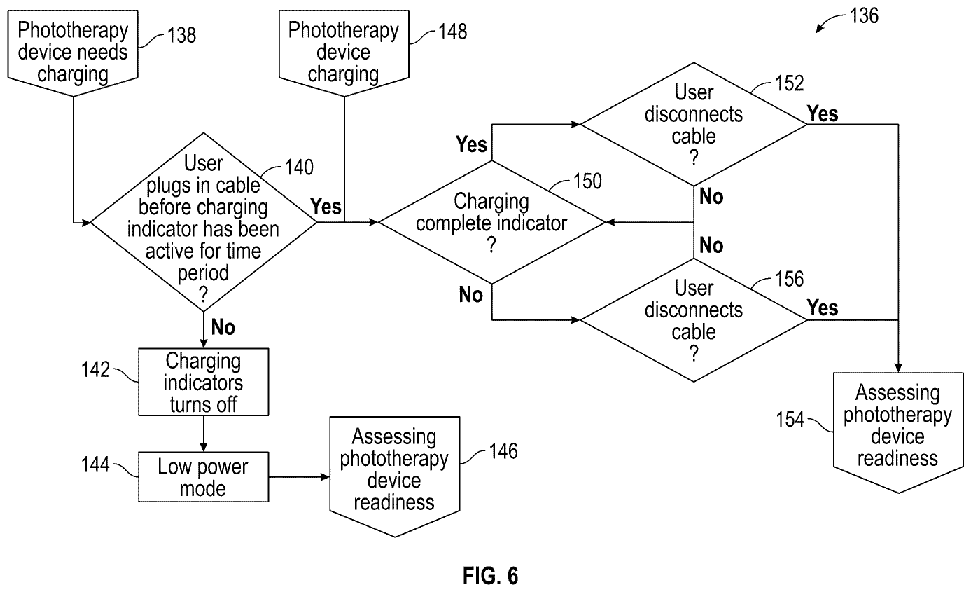

FIG. 6 is a schematic diagram of a process flow 136 during charging of the phototherapy device 10 of FIGS. 1A to 1C. The process flow 136 may occur between step 100 and step 104 of FIG. 4 and/or after step 120 of FIG. 5. If the phototherapy device needs charging (step 138), the charging indicator of step 118 of FIG. 5 may be active for a time period. The logic may then determine if the user plugs in a charging cable during the time period (step 140). If the device is not plugged in, the charging indicator may turn off (step 142), the phototherapy device may return to low power mode (step 144), and the phototherapy device may revert to the assessing device readiness state (step 146). Accordingly, the logic may return to the process flow 112 of FIG. 5. If the phototherapy device is plugged in for charging (step 148), a charging complete indicator (step 150), such as a pulsing light of a third color that is different from the first and second colors of FIG. 5, may indicate to the user to disconnect the charging cable (step 152). At this point, the phototherapy device may revert to the assessing device readiness state (step 154) and return to the process flow 112 of FIG. 5. If the user does not disconnect the cable, the charging complete indicator may remain active (step 150). If the user disconnects the charging cable before the charging is complete (step 156), the device may also revert to the assessing device readiness state (step 154) and return to the process flow 112 of FIG. 5.

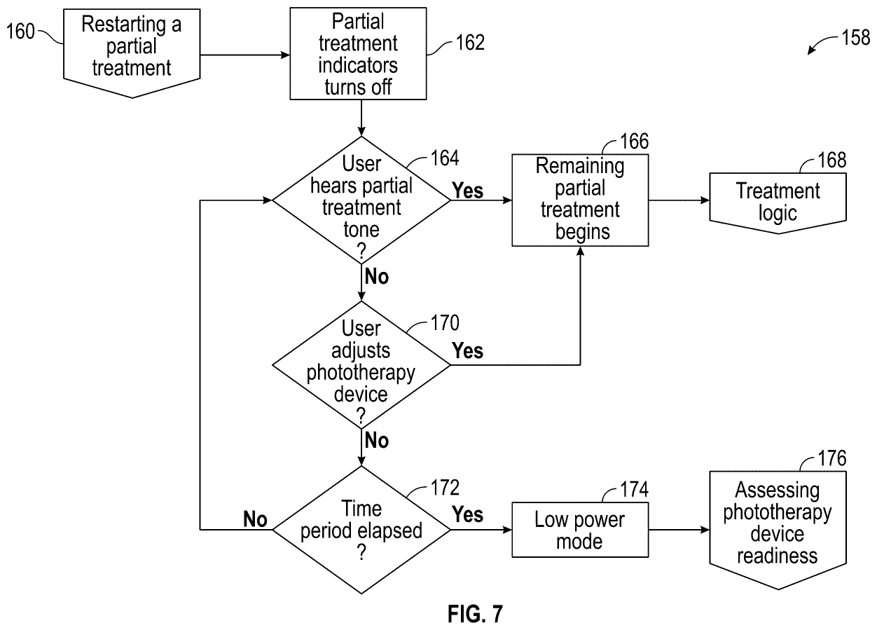

FIG. 7 is a schematic diagram of a process flow 158 for resuming a partial treatment for the phototherapy device 10 of FIGS. 1A to 1C. The process flow 158 of FIG. 7 may occur during one or more of steps 122, 124, 126, and/or 128 of FIG. 5. For initiation of partial treatment (step 160), the partial treatment indicator may turn off (step 162) may turn off, and the speaker 90 of FIG. 3 may output an audible partial treatment tone (step 164), such as two ascending tones. The remaining partial treatment may resume (step 166), and the phototherapy device may follow a treatment logic (step 168). If the partial treatment tone is not provided, the user may adjust the phototherapy device (step 170) and then progress to the resuming partial treatment (step 166) upon the phototherapy device detecting motion and/or placement. If a time period has elapsed without detecting motion (step 172), low power mode may resume (step 174), and the phototherapy device may revert to the assessing device readiness state (step 176) and return to the process flow 112 of FIG. 5.

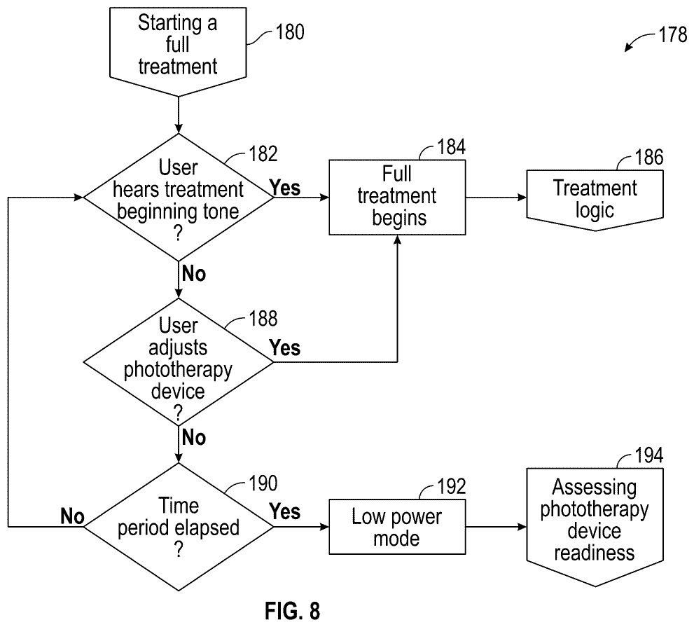

FIG. 8 is a schematic diagram of a process flow 178 for starting a full treatment for the phototherapy device 10 of FIGS. 1A to 1C. The process flow 178 of FIG. 8 may occur during one or more of steps 130, 132, 134, and/or 128 of FIG. 5. For initiation of a full treatment (step 180), the speaker 90 of FIG. 3 may output a beginning treatment tone (step 182), such as a series of countdown tones. The full treatment may begin (step 184), and the phototherapy device may follow a treatment logic (step 186). If the beginning treatment tone is not provided, the user may adjust the phototherapy device (step 188) and then progress to the full treatment (step 184) upon the phototherapy device detecting motion and/or placement. If a time period has elapsed without detecting motion (step 190), low power mode may resume (step 192), and the phototherapy device may revert to the assessing device readiness state (step 194) and return to the process flow 112 of FIG. 5.

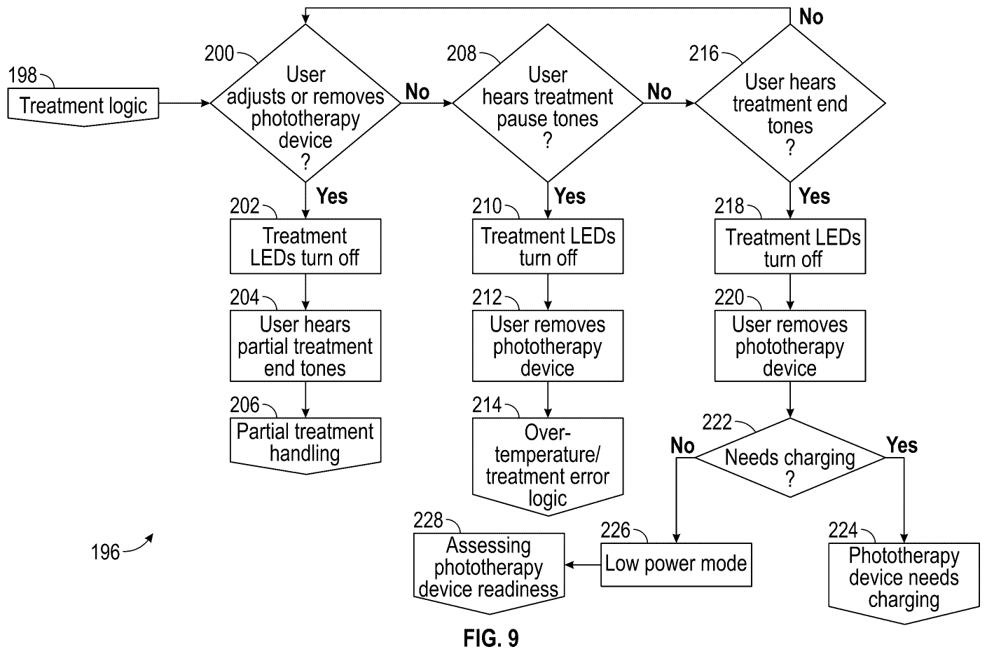

FIG. 9 is a schematic diagram of a process flow 196 for treatment logic for the phototherapy device 10 of FIGS. 1A to 1C. The process flow 196 of FIG. 9 may occur at step 168 of FIG. 7 and/or at step 186 of FIG. 8. In FIG. 9, the phototherapy device begins treatment logic (step 198). During treatment, the phototherapy device may monitor if the user adjusts and/or removes the phototherapy device (step 200) by way of the proximity sensor and/or accelerometer. If adjustment and/or removal occurs that interrupts treatment, then the treatment light-emitting devices turn off (step 202), a partial treatment tone, such as two descending tones, may be provided (step 204), and the logic may revert to partial treatment handling (step 206) and further revert to the process flow 158 of FIG. 7 and/or step 122 of FIG. 5. If the phototherapy device is not adjusted or removed, treatment may continue.

In certain situations, such as an over-temperature and/or an error state, a treatment pause tone (step 208) may be initiated. The phototherapy device may then turn off the treatment light-emitting devices (step 210), the user may remove the phototherapy device (step 212), and the logic may progress to over-temperature and/or treatment error logic (step 214). If the full treatment continues to completion without incident, the phototherapy device may provide a treatment end tone (step 216), such as three repeated tones, the treatment light-emitting devices may turn off (step 218), and the user may remove the phototherapy device (step 220). The logic may then determine if charging is needed (step 222). If charging is needed (step 224), the phototherapy device may progress to the process flow 136 of FIG. 6. If charging is not needed, low power mode may resume (step 226) and the phototherapy device may revert to the assessing device readiness state (step 228) and return to the process flow 112 of FIG. 5.

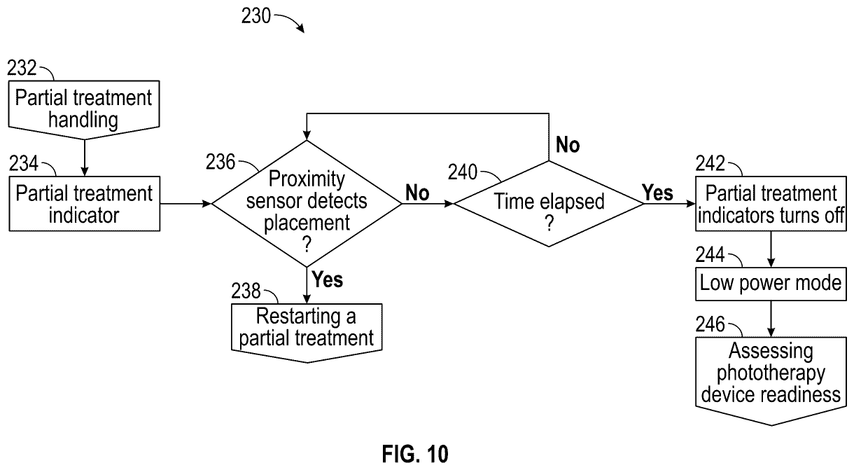

FIG. 10 is a schematic diagram of a process flow 230 for partial treatment logic for the phototherapy device 10 of FIGS. 1A to 1C. The process flow 230 of FIG. 10 may occur at step 206 of FIG. 9. After a full treatment is interrupted, the phototherapy device may progress to partial treatment handling (step 232) and display the partial treatment indicator (step 234). The phototherapy device may then monitor for placement by way of the proximity sensor (step 236). If suitable placement is detected, partial treatment may be restarted (step 238) and the phototherapy device may progress to the process flow 158 of FIG. 7. The phototherapy device will wait for suitable placement for a time period. If the time period has elapsed (step 240), the partial treatment indicator will turn off (step 242), low power mode may resume (step 244) and the phototherapy device may revert to the assessing device readiness state (step 246) and return to the process flow 112 of FIG. 5.

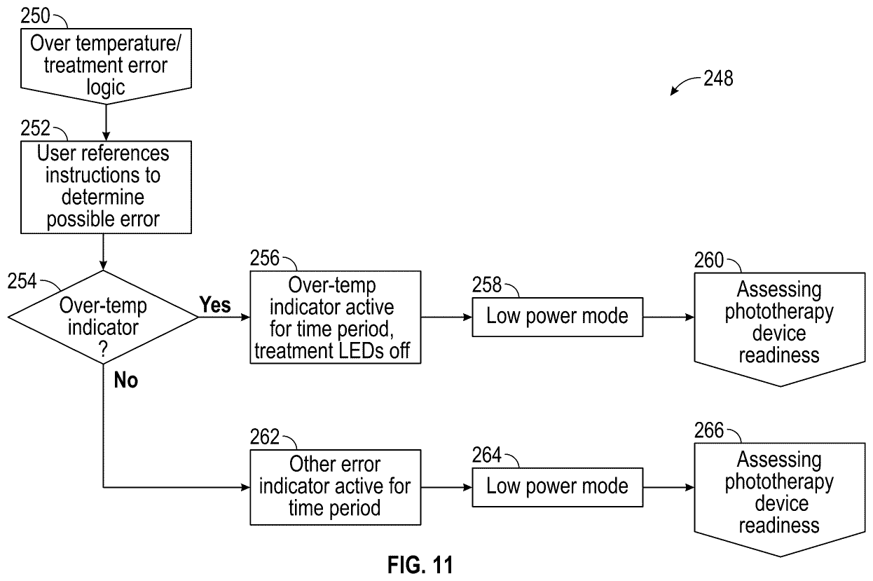

FIG. 11 is a schematic diagram of a process flow 248 for over-temperature and/or error logic for the phototherapy device 10 of FIGS. 1A to 1C. The process flow 248 of FIG. 11 may occur at step 214 of FIG. 9. After treatment is interrupted for an over-temperature and/or an error condition, the phototherapy device may progress to over-temperature and/or treatment error logic (step 250). In certain embodiments, the user may optionally reference instructions to determine a possible error (step 252). In certain embodiments, the device may display an over-temperature indicator (step 254), such as light of a fourth color different from other indicator colors. If the over-temperature indicator is active, the treatment light-emitting devices may turn off for a time period (step 256), such as ten minutes, or twenty minutes, during which the user may not operate the phototherapy device. After the time period has passed to allow sufficient cooling, the phototherapy device may enter low power mode (step 258), revert to the assessing device readiness state (step 260), and return to the process flow 112 of FIG. 5. If the over-temperature indicator is not displayed, an error indicator, such as a solid or flashing light of a fifth color, may optionally be displayed for a time period (262), such as two minutes, or five minutes, or ten minutes. After this time period, the phototherapy device may enter low power mode (step 264), revert to the assessing device readiness state (step 266), and return to the process flow 112 of FIG. 5.

It is contemplated that any of the foregoing aspects, and/or various separate aspects and features as described herein, may be combined for additional advantage. Any of the various embodiments as disclosed herein may be combined with one or more other disclosed embodiments unless indicated to the contrary herein.

Those skilled in the art will recognize improvements and modifications to the preferred embodiments of the present disclosure. All such improvements and modifications are considered within the scope of the concepts disclosed herein and the claims that follow.

What is claimed is:

1. A phototherapy device comprising:
an array of light-emitting devices;
a control printed circuit board configured to control operation of the array of light-emitting devices;
a first sensor; and
a second sensor, the first sensor configured to provide a first signal that directs the control printed circuit board to respond to a second signal from the second sensor.

2. The phototherapy device of claim 1, wherein the control printed circuit board is configured to electrically activate the array of light-emitting devices based on the second signal.

3. The phototherapy device of claim 1, wherein the first sensor comprises an accelerometer, and the second sensor comprises a proximity sensor.

4. The phototherapy device of claim 3, wherein the accelerometer is positioned on the control printed circuit board and the proximity sensor is positioned away from the control printed circuit board.

5. The phototherapy device of claim 4, further comprising a flexible substrate, wherein the control printed circuit board and the proximity sensor are on different portions of the flexible substate.

6. The phototherapy device of claim 5, wherein:
the flexible substrate comprises a proximal surface and a distal surface that is opposite the proximal surface, the flexible substate being configured for positioning along a scalp of a user such that the proximal surface is closer to the scalp than the distal surface;
the array of light-emitting devices is on the proximal surface;
the control printed circuit board is on the distal surface; and
the proximity sensor is on the proximal surface.

7. The phototherapy device of claim 6, further comprising a light-transmissive layer on the proximal surface of the flexible substrate, the light-transmissive layer configured to transmit at least some light emissions generated by the array of light emitting devices.

8. The phototherapy device of claim 1, further comprising an electronic connection port configured to receive an external power source, wherein the control printed circuit board is configured to ignore the first sensor until after the external power source is connected to the electronic connection port.

9. The phototherapy device of claim 1, wherein the array of light-emitting devices is configured to generate light having a first peak wavelength in a range from 600 nanometers (nm) to 700 nm.

10. The phototherapy device of claim 1, wherein the array of light-emitting devices is configured to generate light having a first peak wavelength in a range from 615 nm to 635 nm and a second peak wavelength in a range from 650 nm to 670 nm.

11. A method comprising:
detecting a first signal from a first sensor within a phototherapy device;
directing a control printed circuit board within the phototherapy device to respond to a second signal from a second sensor within the phototherapy device after receiving the first signal; and
electrically activating an array of light-emitting devices after receiving the second signal.

12. The method of claim 11, further comprising connecting an external power source to charge the phototherapy device, wherein the phototherapy device is in a shipping mode before connecting the external power source, and the phototherapy device is configured to ignore the first sensor and the second sensor during the shipping mode.

13. The method of claim 12, wherein after connecting the external power source, the phototherapy device exits the shipping mode and begins a low power mode that will respond to the first sensor.

14. The method of claim 13, wherein after receiving the first signal during the low power mode, the phototherapy device exits the low power mode and begins monitoring for the second signal for a time period.

15. The method of claim 14, further comprising returning to the low power mode if the phototherapy device does not receive the second signal during the time period.

16. The method of claim 14, wherein the phototherapy device determines if a previous treatment was ended prematurely as a partial treatment, and resumes the partial treatment after receiving the second signal during the time period.

17. The method of claim 11, wherein the first sensor comprises an accelerometer, and the second sensor comprises a proximity sensor.

18. The method of claim 11, further comprising turning off the array of light-emitting devices in response to an over-temperature condition or an error condition.

19. The method of claim 11, wherein the array of light-emitting devices is configured to generate light having a first peak wavelength in a range from 600 nanometers (nm) to 700 nm.

20. The method of claim 11, wherein the array of light-emitting devices is configured to generate light having a first peak wavelength in a range from 615 nm to 635 nm and a second peak wavelength in a range from 650 nm to 670 nm.
